(19) United States
(12) Patent Application Publication
(10) Pub. No.: US 2025/0352141 A1
(43) Pub. Date: Nov. 20, 2025

(54) METHODS AND SYSTEMS FOR MONITORING INTERSTITIAL LUNG DISEASE PATIENT EVENTS IN REAL-TIME

(71) Applicant: Evinova AG, Baar (CH)

(72) Inventors: Elisabeth Carine PIAULT-LOUIS, San Francisco, CA (US); Emmette Ramsey HUTCHISON, Austin, TX (US); Alicyn Katherine CAMPBELL, Bennington County, VT (US)

(21) Appl. No.: 19/208,403

(22) Filed: May 14, 2025

Related U.S. Application Data
(60) Provisional application No. 63/647,792, filed on May 15, 2024.

(51) Int. Cl. A61B 5/00; A61B 5/1455; G16H 10/60

(52) U.S. Cl. CPC A61B 5/4848; A61B 5/0004; A61B 5/14551; A61B 5/7275; A61B 5/74; G16H 10/60

(57) ABSTRACT

A patient at risk of lung disease due to a prescribed treatment may be remotely monitored. A cloud server may receive data collected from a patient and analyze the data to determine if the patient shows signs of onset or worsening interstitial lung disease. The cloud server may transmit the analyzed data to a healthcare provider in real-time or near real-time to provide timely symptom management to the patient.

### BACKGROUND

Interstitial Lung Disease (ILD) refers to a large group of diseases that cause scarring (i.e., fibrosis) of the lungs, which leads to shortness of breath and impacts delivery of oxygen to the bloodstream. ILD is a term used for more than 44 International Classification of Diseases (ICD) codes and indicates various case presentations of lung inflammation. ILD is a side effect of interest across a myriad of treatments including, but not limited to, oncology therapies. ILD is typically misdiagnosed or diagnosed through exclusion, which leads to significant resource utilization, delay in treatment initiation, and higher morbidity. Across tumor types and therapies, many efficacious treatments considered to increase the risk of ILD are not used to their maximum therapeutic benefit because of the increased risk of developing ILD, which results in missed opportunities to improve clinical outcomes for patients.

A specific ILD of interest is pneumonitis. Pneumonitis is a common and serious adverse event that impacts optimal treatment of non-small cell lung cancer (NSCLC). The risk of pneumonitis varies across NSCLC patients depending on treatment regimens, including prior therapies. Up to 18% of NSCLC patients are affected by pneumonitis, which has high morbidity, progresses within days, and is associated with increased resource use, decreased treatment benefit, and mortality. Additionally, the risk of high-grade pneumonitis is exacerbated by limited awareness of pneumonitis by patients and inability of health care providers (HCPs) to timely manage pneumonitis.

Pneumonitis presents itself in distinct patterns but requires vigilant attention to symptoms by patients and HCPs. For these reasons, many HCPs hesitate to treat patients with the most efficacious treatments for NSCLC. Specifically, HCPs hesitate to start patients on treatments that include pneumonitis risk on labels, which has lead up to 20% of NSCLC patients to not be initiated on Standard of Care (SoC) or novel treatments for NSCLC. Additionally, when a patient is treated with a SoC or novel treatment, early detection of pulmonary changes is essential as it enables early management of pneumonitis through diagnosis and treatment.

Because of the above issues relating to diagnosis and treatment of pneumonitis and other ILDs, there is a need for a system that determines and monitors a patient's treatment and symptoms in a prompt and efficacious manner to facilitate effective NSCLC and ILD treatment by surfacing patients at risk of ILD.

### BRIEF SUMMARY

Methods, systems, and computer readable mediums for remotely monitoring a patient at risk of lung disease due to a prescribed treatment are provided herein. The method includes receiving, by one or more processors, patient data collected from the patient prior to prescribing treatment of cancer in the patient, wherein the treatment is determined to cause a lung disease; identifying, by the one or more processors, a risk factor of lung disease in the patient by providing the patient data to a rule-based algorithm; while the patient receives treatment, receiving, by the one or more processors, physiological data of the patient from a patient device, wherein the physiological data comprises oxygen saturation data measured by a pulse-oximeter optionally coupled to the patient device and subjective patient data from the patient device corresponding to patient-reported experience; determining, by the one or more processors, a status change of the patient by providing the risk factor of lung disease, the physiological data, and the subjective patient data received during treatment of the patient to a longitudinal algorithm, wherein the status change indicates suspicion of the lung disease in the patient; transmitting, by the one or more processors, a notification of the status change of the patient to a health care provider device.

Further embodiments, features, and advantages of the present invention, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate aspects of the present invention and, together with the description, further serve to explain the principles of the invention and to enable a person skilled in the pertinent art to make and use the invention.

FIG. 1 illustrates a block diagram of a system for remotely monitoring a patient being treated for a disease, according to some aspects.

FIG. 2A illustrates an example of an interface of a patient application operating on the patient's device, according to some aspects. The example interface shows examples of physiological measurements or subjective patient data that could be obtained or provided.

FIG. 2B illustrates an example of an interface of a patient application operating on the patient's device, according to some aspects. The example interface shows how a patient could provide a subjective rating of the severity of a symptom on a scale of 0 to 10.

FIG. 2C illustrates an example of an interface of a patient application operating on the patient's device, according to some aspects. The example interface shows how the application could interface with a medical device communicating with the patient's device to obtain physiological data.

FIG. 3A illustrates an example of an interface for a healthcare provider that provides an overview of multiple patients, according to some aspects.

FIG. 3B illustrates an example of an interface for a health care provider for a single patient, according to some aspects.

FIG. 4 illustrates a method for remotely monitoring a patient at risk of lung disease due to a prescribed treatment, according to some aspects.

FIG. 5 illustrates a block diagram of example components of a computer system, according to aspects of the present disclosure.

FIG. 6 illustrates examples of baseline rules that could be used to calculate a patient's risk factor.

Aspects of the present invention will be described with reference to the accompanying drawings. The drawing in which an element first appears is typically indicated by the leftmost digit(s) in the corresponding reference number.

### DETAILED DESCRIPTION

Diagnosis of ILD requires significant resources and is often delayed because ILD is a disease of exclusion. The delay is further compounded because patients often do not timely and accurately report symptoms associated with ILD. Because healthcare providers (HCPs) have limited options to proactively monitor patients at risk of interstitial lung disease (ILD), many patients do not receive optimal treatment for non-small cell lung cancer (NSCLC). When a patient is prescribed the Standard of Care (SoC) treatment for NSCLC, HCPs must continuously follow-up with patients that are on-treatment for potential ILD events via clinical visits. These visits are time intensive and require regular clinical re-assessment since patients must stop taking effective treatments for NSCLC to treat ILD symptoms should the patient present with ILD. Because treatment of ILD includes discontinuing NSCLC treatment, high grade ILD prevents optimal NSCLC treatment outcomes and limits re-initiation of treatment as previous history of ILD is an established risk factor for developing subsequent ILD. Up to 15% of patients experience ILD related discontinuations of treatment and about two-thirds of the discontinuations become permanent. Therefore, early and accurate detection allows NSCLC patients to continue or quickly resume treatment regimens, which leads to better clinical outcomes and quality of life. Consequently, a system that mitigates the risk of high-grade ILDs through timely management and risk assessment may lead to effective treatment for patients undergoing NSCLC treatment and reduce the risks associated with developing ILD.

Provided herein is a digital solution that supports HCPs treating NSCLC patients while remotely monitoring patients for symptoms of ILD. The remote monitoring system collects symptoms and physiological data from patients receiving NSCLC treatment, analyzes the information compared to the patient's baseline status and risk factors, and provides timely notifications to the HCP that the patient is exhibiting signs of ILD. This digital solution allows HCPs to remotely monitor patients and their pulmonary health, which increases HCP's confidence and ability to manage patients at risk of ILD.

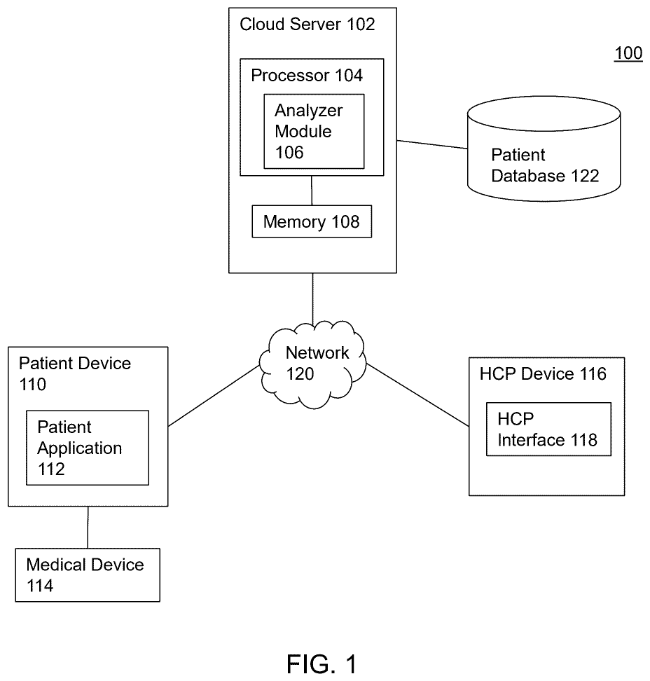

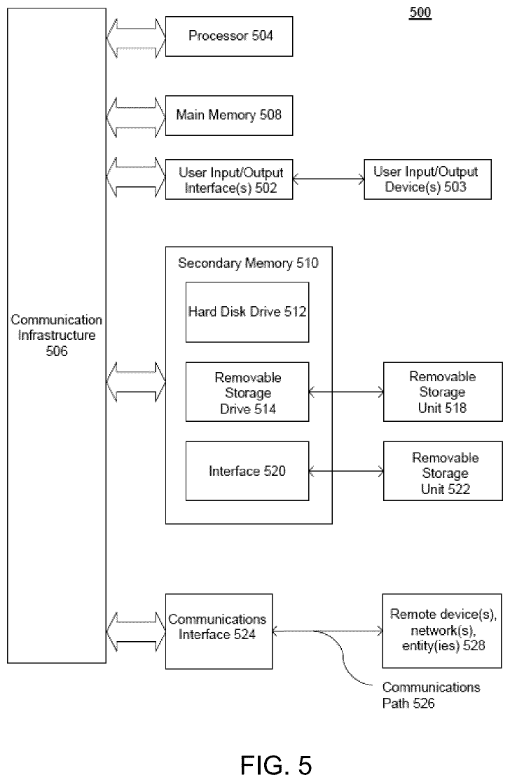

FIG. 1 illustrates an example system 100 for remotely monitoring a patient being treated for a disease when the treatment presents risks of causing lung disease, although other systems with similar functionality may alternatively be used. In some aspects, the patient may be receiving treatment for NSCLC. System 100 includes a cloud server 102, which is a private and secure cloud server configured to process and store patient data in a cloud environment. Cloud server 102 may be communicatively coupled to patient device 110 and HCP device 116 via one or more networks 120. Cloud server 102 includes a processor 104 and memory 108. Processor 104 may be a processing device, such as computer system 500 described with reference to FIG. 5 below. Processor 104 may include analyzer module 106 which is configured to perform rule-based and longitudinal algorithms on collected data, as described in further detail below.

Patient database 122 is communicatively coupled to cloud server 102. Data in patient database 122 includes, but is not limited to, electronic case report forms (eCRF), electronic medical records (EMRs), medical imaging, medical history, demographics, or pulmonary function data of a plurality of patients. Data in patient database 122 may be provided to analyzer module 106 to identify the risk factor of a patient developing ILD while using a specific treatment.

Network 120 may be of any suitable type, including individual connections via the internet such as cellular or WiFi networks. In some embodiments, network 120 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, or LAN. Because the information transmitted may be personal or confidential, security concerns may dictate one or more of these types of connections be encrypted or otherwise secured.

Network 120 may comprise any type of computer networking arrangement used to exchange data. For example, network 120 may be the internet, a private data network, virtual private network using a public network, and/or other suitable connection(s) that enables components in system 100 to send and receive information between the components of system 100. Network 120 may also include a public switched telephone network (“PSTN”) and/or a wireless network.

Patient device 110 is communicatively coupled to cloud server 102 via network 120. Patient device 110 can be any type of computing device, including a laptop, a desktop, a smartphone, a tablet computer, or a wearable computer (such as a smartwatch or an augmented reality or virtual reality headset). Not shown, patient device 110 also includes a processor and persistent, non-transitory and volatile memory. The processors can include one or more central processing units, graphic processing units, or any combination thereof. Patient device 110 may be a processing device, such as computer system 500 described with reference to FIG. 5 below.

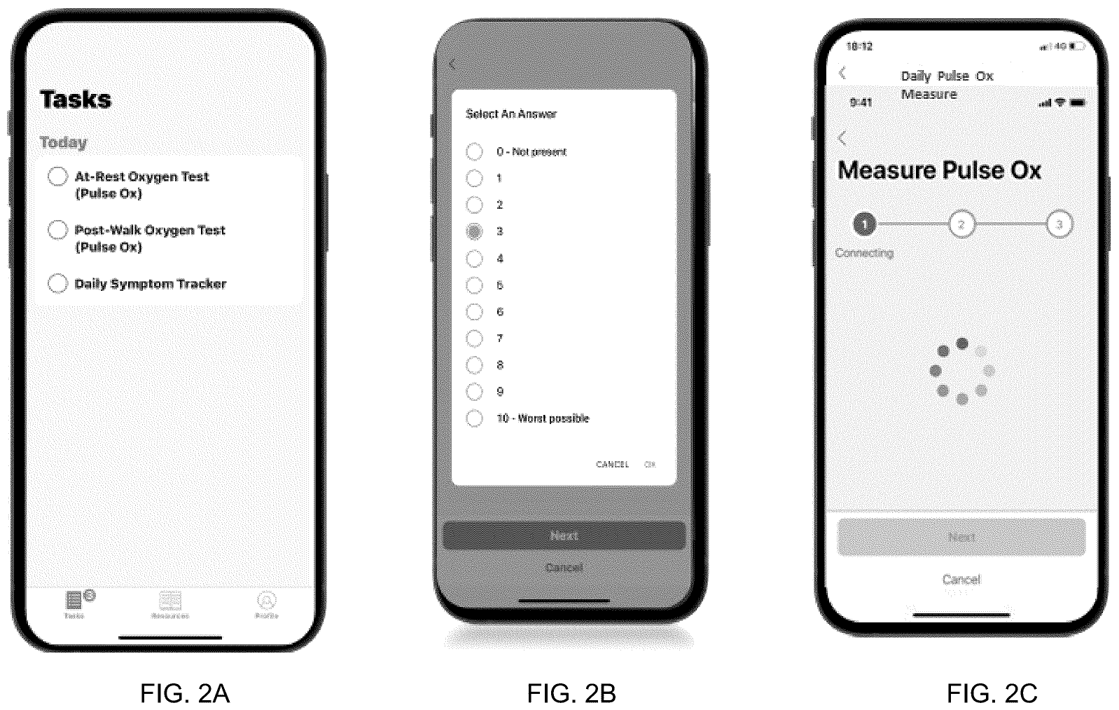

Patient device 110 includes a patient application 112. Patient application 112 may be a web application downloaded from cloud server 102. Patient application 112 provides a high useability, low-burden digital experience with behavioral nudges (i.e., notifications) to collect, analyze, and transmit patient symptom and physiological data in real-time. The patient-friendly multi-modal digital data-capture interface guides patients to recognize and report signs and symptoms consistent with ILD. Exemplary functions of patient application 112 are illustrated in FIGS. 2A-2C. FIG. 2A displays an example representation of a daily task list to the patient that provides specific behavioral nudges for the patient to input and collect the patient's physiological data and/or subjective patient data. In some configurations, a behavioral nudge for each task may appear as a notification on the patient's device. The one or more processors 104 of the cloud server 102 and/or the HCP device 116 may push or transmit a notification via the network 120 to the patient device 110. The notification may correspond to a text message with a link that when selected opens the patient application 112 to show the task list provided in FIG. 2A. FIG. 2B displays an example of a symptom tracker that allows the patient to input their symptoms to be analyzed by analyzer module 106. FIG. 2C displays an example interface for collecting the patient's oxygen saturation using medical device 114. Information provided by the patient may be received as subjective patient data (e.g., Patient Reported Outcome (PRO)). Such data may include, for example, a patient reporting a shortness of breath, a cough, tightness in the chest. The severity of the subjective patient reported data may be indicated by scale such as shown in FIG. 2B.

Medical device 114 is any device that may be coupled to patient device 110. In some aspects, medical device 114 is a pulse oximeter device with Bluetooth capability (e.g., Massimo MightySat Rx 9909 or Massimo MightySat Rx 9907) that enables connection to patient device 110. In some aspects, medical device 114 collects a patient's oxygen saturation data and transmits that data to patient application 112. In some instances, the medical device 114 does not require any input from the patient device 110 for the medical device's 114 functionality. For example, the medical device 114 may be a wearable device that automatically collects and/or transmits patient physiological data continuously, at predetermined temporal intervals (e.g., monthly, weekly, daily, every 4 hours), or at predetermined event intervals (e.g., patient heart rate, glucose, or temperature has reached a certain level). As another example, the medical device 114 may contain an interface that allows the patient to activate the medical device's 114 functionality. The medical device 114 may update the information in the symptom tracker, which then can be relayed to the HCP device 116 and/or the cloud server 102 via the network 120. In some configurations, the patient device 110 is a smart watch that can, for example, collect and/or transmit the patient's oxygen saturation data, pulse, or other medically relevant information. As noted above, the medical device 114 may connect directly to the patient device 110 or indirectly to the patient device 110, the HCP device 116, and/or cloud server 102 via the network 120.

HCP device 116 is communicatively coupled to cloud server 102 via network 120. HCP device 116 can be any type of computing device, including a laptop, a desktop, a smartphone, a tablet computer, or a wearable computer (such as a smartwatch or an augmented reality or virtual reality headset). Not shown, HCP device 116 also includes a processor and persistent, non-transitory and volatile memory. The processors can include one or more central processing units, graphic processing units or any combination thereof. HCP device 116 may be a processing device, such as computer system 500 described with reference to FIG. 5 below.

HCP device 116 includes HCP interface 118. HCP interface 118 may be a web application downloaded from cloud server 102, or may be a native application running on HCP device 116. HCP interface 118 may be configured to be executed by a respective web browser. In some aspects, HCP interface 118 is an iterative dashboard to monitor signs and symptoms consistent with ILD in light of a patient's existing risk factor. HCP interface 118 uses analytic-enabled alerts of status changes of a patient to ensure timely insight and management by the HCP. Additionally, HCP interface 118 provides a patient-level data display to provide full transparency of a patient's status for quick, data-driven decision making by the HCP.

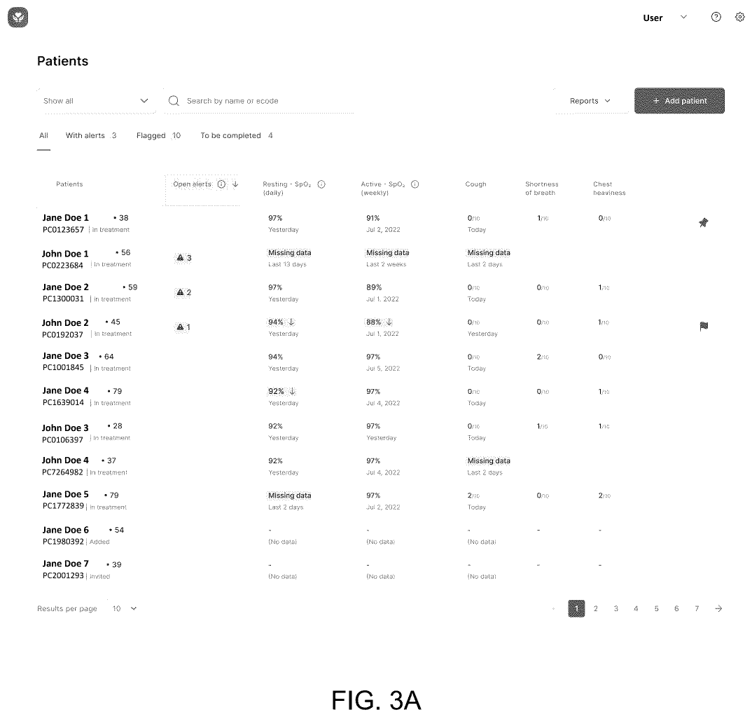

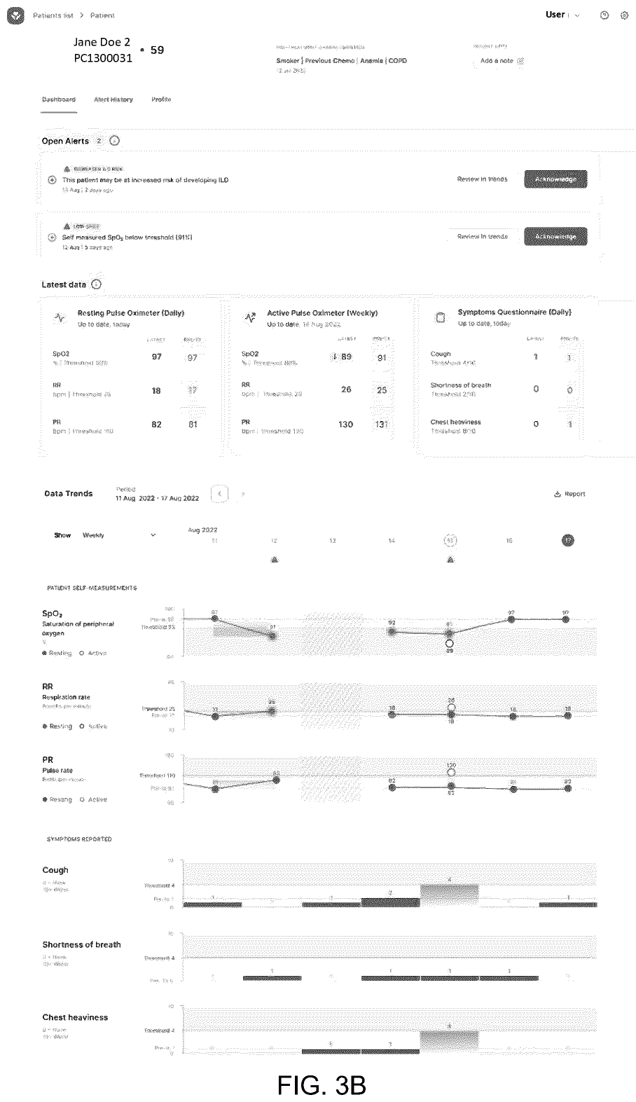

FIG. 3A illustrates an example of an interface for a healthcare provider that provides an overview of multiple patients. Each row in the example interface corresponds to an individual patient. The interface can show an indication of some of the physiological data for the patient at multiple intervals (e.g., hourly, daily, weekly). It can show patient reported experiences such as cough, shortness of breath, chest heaviness. It can also provide an indication of outstanding alerts for each patient (e.g., under the column “Open alerts” in this example). These alerts may correspond to outstanding tasks for the patient such as providing physiological data from a medical device or reporting the patient's experience. In some cases, the interface may show a flag (e.g., for patient John Doe 2 at far right of row). The flag may be generated by the methods disclosed herein and indicate to the HCP that the patient may be experiencing ILD. The interface may also permit the HCP to pin specific patients to the top of the list such as Jane Doe 1. The HCP may wish to closely monitor a patient and manually pin the patient. The system may pin a patient to the top of the list where further action or monitoring is warranted by the HCP. The interface shown in FIG. 3A may be dynamic and/or configured to show specific types of information. For example, one HCP may wish to hide the symptoms for “Cough” while another may wish to only show those patients who are pinned, have open alerts, or have a flag.

FIG. 3B illustrates an example of an interface for a health care provider for a single patient, according to some aspects. The single patient interface provides a more detailed view of the patient's history of physiological and patient reported data, medical history, and alerts. For example, a HCP can review any trends associated with the physiological data or patient reported data over a selected time frame (e.g., a week, a month, a year). The information shown on the patient-specific interface in the example provided in FIG. 3B is also configurable.

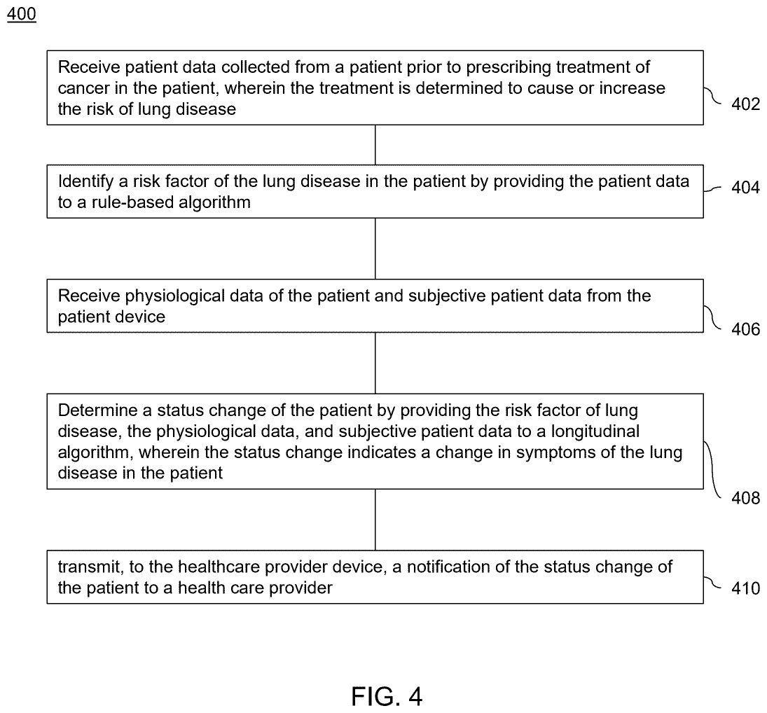

FIG. 4 illustrates a method 400 for remotely monitoring a patient at risk of ILD due to a prescribed treatment. The remote monitoring may be used to determine a patient's potential risk factors and alert a HCP of symptoms and signs of ILD in a patient using the remote monitoring system. Method 400 will be described with reference to system 100 illustrated in FIG. 1, although other systems with similar functionality may alternatively be used. While the present disclosure uses ILD as an example throughout, in some aspects method 400 may similarly be used to monitor a patient at risk for other diseases caused by a prescribed treatment.

At 402, cloud server 102 receives patient data collected from a patient prior to the patient beginning a treatment to the patient for a disease. In some aspects, the disease may be a particular type of cancer (e.g., non-small cell lung cancer) or non-cancer. The patient data may include a patient's medical history, demographics, and/or pulmonary function.

The pulmonary function may include physician's notes from an HR-CT scan of the patient. The patient data may additionally include imaging, if available, which may be imaging from an X-ray, computed tomography (CT) scan, magnetic resonance imaging (MRI), or other known medical imaging mechanisms. In some aspects, the patient data may be stored in memory 108. In another aspect, the patient data may be stored in patient database 122. In another aspect, the patient data may be received directly from HCP device 116. In some aspects, at least a portion of the patient data is contained in an eCRF or EMR.

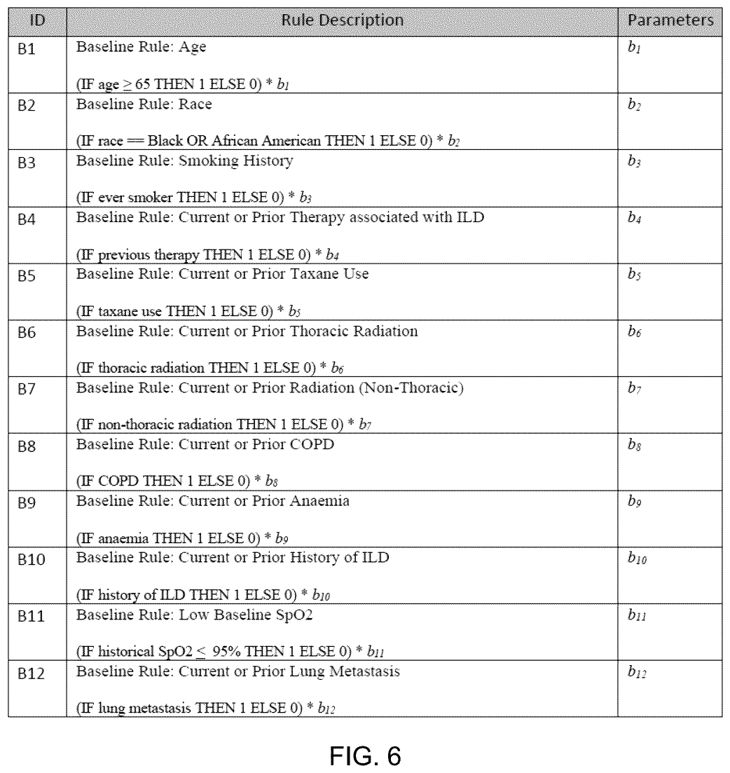

Examples of such patient data include, for example, patient age, patient race, whether the patient has a current or prior therapy associated with ILD, whether patient has a current or prior taxane use, whether the patient has a current or prior thoracic or non-thoracic radiation treatment, whether the patient has current or prior chronic obstructive pulmonary disease (COPD), whether the patient has a current or prior anemia, whether the patient has a current or prior ILD, a baseline SpO2, whether the patient has a current or prior lung metastasis, whether the patient is a current or prior user of cigarettes, whether the patient has currently or previously had asthma or symptoms from an illness affecting the lungs (e.g., pneumonia, COVID-19). An example of the patient data is provided in FIG. 6.

The treatment described at 402 may be a treatment that has been determined to cause or increase the risk of lung disease. Specifically, the lung disease may be ILD. ILD refers to a large group of lung diseases which includes, but is not limited to, acute interstitial pneumonitis; acute respiratory distress syndrome; acute respiratory failure; air-space consolidation; allergic eosinophilia; alveolar proteinosis; alveolitis; alveolitis allergic; alveolitis necrotizing; architectural distortion; autoimmune lung disease; bronchiolitis; chronic interstitial (CIP); combined pulmonary fibrosis and emphysema; diffuse alveolar damage; drug-induced interstitial lung disorder; eosinophilia myalgia syndrome; eosinophilic granulomatosis with polyangiitis; eosinophilic pneumonia; eosinophilic pneumonia acute; eosinophilic pneumonia chronic; granulomatous pneumonitis; ground-glass attenuation; idiopathic interstitial pneumonia; idiopathic pneumonia syndrome; idiopathic pulmonary fibrosis; interlobular septal thickening; interstitial lung disease; interstitial pneumonia; interstitial pulmonary disease; intralobular reticular opacity; lung infiltration; lymphangitic carcinomatosis; necrotizing bronchiolitis; nodular opacities; non-septal linear opacity; obliterative bronchiolitis; organizing pneumonia; pleural effusion; pneumonitis; previous tuberculosis; progressive massive fibrosis; pulmonary emphysema; pulmonary fibrosis; pulmonary necrosis; pulmonary radiation injury; pulmonary sarcoidosis; pulmonary toxicity; pulmonary vasculitis; radiation alveolitis; radiation fibrosis; radiation pneumonitis; respiratory failure; restrictive pulmonary disease; rheumatoid lung sarcoidosis; scarring or inflammation of interstitium; small airways disease; thickening of bronchovascular bundles; traction bronchiectasis; transfusion-related acute lung injury; and pneumonia.

In some aspects, treatments that are suspected to cause ILD include, but are not limited to, abatacept; adalimumab; afatinib; amiodarone; atezolizumab; avelumab; bleomycin; cemiplimab; certolizumab; certolizumab-pegol; docetaxel; durvalumab; erlotinib; etanercept; everolimus; enhertu; fluorouracil, epirubicin, and cyclophosphamide (FEC) followed by weekly paclitaxel; gefitinib; gemcitabine; gemcitabine and vinorelbine; golimumab; infliximab; ipilimumab; leflunomide; methotrexate; nab-paclitaxel; nitrofurantoin; nivolumab; osimertinib; paclitaxel; pembrolizumab; rituximab; tocilizumab; select mTOR inhibitors; sirolimus; and temsirolimus.

At 404, the patient data is provided to analyzer module 106. Analyzer module 106 identifies a risk factor of the patient developing lung disease due to treatment for the primary disease using a rule-based algorithm. The risk factor, S, provides a HCP with the risk each patient has of developing ILD. The risk factor is based upon a patient's specific pre-dispositions that would affect the signs and symptoms that indicate the patient is developing lung disease. Performing this analysis at treatment initiation provides a baseline for future analysis.

The risk factor may be based on the sum of the patient data, computed as R, which may include some or all of the factors shown in FIG. 6. For example, R, may be the sum of each baseline rule B1 to B12 of FIG. 6, or a selected portion of B1 to B12 (e.g., any combination of two or more of the baseline rules B1 to B12), or other baseline rules for the disease not shown in FIG. 6. A predetermined offset value is used as a to compute the S. The value of any one baseline parameter b₁ to b₁₂ provided in FIG. 6 may be 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, or any hundredth or thousandth increment between 0 and 1. In some configurations, and as an example, the baseline parameters have the following specific values: b₁=1.0, b₂=0.25, b₃=1.0, b₄=1.0, b₅=0.5, b₆=0.5, b₇=0.5, b₈=0.5, b₉=0.5, b₁₀=1.0, b₁₁=1.0, and b₁₂=0.5.

The sum, R, and the predetermined value of a may be used to calculate S, the risk factor, using the following equation:

$$S = \frac{{e}^{R - a}}{1 + {e}^{R - a}} \qquad Eqn . 1$$

The value of α is a constant. The value of a may be in a range of 0 to 10, 0 to 9, 0 to 8, 0 to 7, 0 to 6, 0 to 5, 0 to 4, 0 to 3, 1 to 10, 1 to 9, 1 to 8, 1 to 7, 1 to 6, 1 to 5, 1 to 4, 1 to 3, 2 to 10, 2 to 9, 2 to 8, 2 to 7, 2 to 6, 2 to 5, 2 to 4, or 2 to 3. The value of a may be proportional to the number of baseline criteria provided. For example if 60 baseline criteria are used instead of the twelve shown in FIG. 6, then the range of values for a may be five times the ranges provided above. The provided ranges include all increments between including the tenths, hundredths, and thousandths. For example, the value of a may be 2.01 to 2.85, 2.10 to 2.50, 2.10 to 2.40, 2.10 to 2.35, 2.10 to 2.30, 2.10 to 2.25, 2.15 to 2.20, or 2.17. The value selected for a may be empirically based for a particular therapy or patient population. The value of Sis dynamic for each patient and is between 0 and 1. For example, as the baseline criteria changes (e.g., a patient might begin smoking), then the value of R, and consequently S, would change.

At 406, while the patient is receiving treatment, cloud server 102 receives physiological data and subjective patient data of the patient from patient device 110 and/or the medical device 114. The physiological data may include one or more of resting oxygen saturation, oxygen saturation at exertion, respiratory rate at rest or during or after exertion, and pulse rate. In some embodiments, the physiological data, such as the oxygen saturation at resting and exertion, may be measured by medical device 114 and automatically transmitted to patient device 110 from medical device 114 or transmitted to the cloud server 102 via the network 120. In some embodiments, the patient may collect and input physiological data from patient device 110 without the supervision or presence of the HCP. In some embodiments, physiological data may be received both automatically from medical device 114 and manually from the patient.

Subjective patient data may include one or more of cough severity, dyspnea severity, chest pain or tightness severity, and/or health-related quality of life measurements. The subjective patient data may be communicated from the patient device 110 to the cloud server 102 via the network 120. The patient may manually input the subjective patient data. As mentioned earlier, the HCP may provide a behavioral nudge to the patient via the patient's device 110 to provide the physiological data or the subjective patient data.

The physiological data may be input multiple times during the course of the patient's treatment. In some aspects, the patient may input their current physiological data daily, weekly, monthly, or throughout any other period of time. The patient may receive a behavioral nudge in patient application 112 to input the physiological data. The behavioral nudge may be provided by cloud server 102 to patient application 112 to suggest that the patient input their physiological data into patient application 112. In some aspects, data received from patient application 112 may be used to determine a patient's engagement with the treatment.

At 408, the physiological data and subjective patient data are provided to analyzer module 106. In some embodiments, analyzer module 106 determines if the patient's status has changed using a longitudinal algorithm. A longitudinal algorithm is one that incorporates change over time in certain physiological data, showing, for example, a patient's response over time to a particular treatment. A longitudinal algorithm according to aspects herein uses changes in the collected physiological parameters or statuses generated therefrom over time, along with population aggregated data, to individualize an action plan for the patient based on the treatment response. For example, a status change may indicate onset or worsening symptoms of lung disease in the patient. The rule-based algorithm operates using a weighted sum over the historical risk factors, physiological signals and patient subjective experience. Each risk factor, physiological signal of interest, patient experience report or change in patient experience of physiological signal is summed on a specified (e.g., hourly, daily, weekly, monthly) basis. If the sum crosses a specific threshold, the HCP is notified of suspected ILD.

An example of how subjective data and physiological data are received, and the associated example rules for the longitudinal algorithm, L1 to L10, are provided in the following table:

| | | |
|---|---|---|
| L1 | Daily Cough PRO fover d [symbol] consecutive days] | [symbol] |
| | [Conditon A] | |
| | (IF today-minus yesterday change > [symbol] ) | |
| | OR | |
| | [Condition B] | |
| | (IF single PRO from current day > [symbol] ) | |
| | AND | |
| | (IE single PRO from yesterday [symbol] ) | |
| | THEN (1 ELSE 0) [symbol] | |
| | Can allow for m [symbol] non-consecutive day(s) of missingness between consecutive readings | |
| L2 | Daily Chest Tightness PRO [over d [symbol] consecutive days] | d [symbol] , t [symbol] , s [symbol] , |
| | [Condition A] | [symbol] , m [symbol] |
| | (IF today-minus-yesterday change > [symbol] ) | |
| | OR | |
| | [Condition B] | |
| | (IF single PRO from current day > [symbol] ) | |
| | AND | |
| | (IF single PRO from yesterday [symbol] ) | |
| | THEN (1 ELSE 0) [symbol] | |
| | Can allow for m [symbol] non-consecutive day(s) of missingness between consecutive readings | |
| L3 | Daily Shortness of Breath PRO [over d [symbol] consecutive days] | d [symbol] , t [symbol] , [symbol] , |
| | [Condition A] | c [symbol] , m [symbol] |
| | (IF today-minus-yesterday change > [symbol] ) | |
| | OR | |
| | Condition B] | |
| | [(IF single PRO from current day > [symbol] ) | |
| | AND | |
| | (IF single PRO from yesterday > [symbol] ) | |
| | THEN ( [symbol] ELSE 0) [symbol] | |
| | Can allow for m [symbol] non-consecutive day(s) of missingness between consecutive readings | |
| L4 | Daily SpO2 at rest [over d [symbol] consecutive days] | d [symbol] , [symbol] , s [symbol] , |
| | [Condition A] | c [symbol] , m [symbol] |
| | (If today-minus-yesterday change < [symbol] ) | |
| | OR | |
| | Condition B] | |
| | [ (IF single Sp [symbol] )2 from current day < [symbol] ) | |
| | AND | |
| | (IF single SpO2 from yesterday < [symbol] ) | |
| | THEN ( [symbol] ELSE O) [symbol] | |
| | Can allow for m [symbol] non-consecutive day(s) of missingness between consecutive readings | |
| L5 | Daily Respiratory Rate at rest [over d [symbol] consecutive days] | d [symbol] , [symbol] , s, [symbol] , |
| | [Condition A] | c [symbol] , m [symbol] |
| | (If today-minus-yesterday change [symbol] ) | |
| | OR | |
| | Condition B] | |
| | [(IF single RR from current day [symbol] ) | |
| | AND | |
| | (IF single RR from yesterday > [symbol] ) | |
| | THEN (1 ELSE 0) [symbol] | |
| | Can allow for m [symbol] non-consecutive day(s) of missingness between consecutive readings | |
| L6 | Daily Pulse Rate at rest [over d [symbol] consecutive days] | d [symbol] , [symbol] , s [symbol] , |
| | [Condition A] | c [symbol] , m [symbol] |
| | (IF today-minus yesterday change > [symbol] ) | |
| | OR | |
| | Condition B] | |
| | [(IF single RR from current day > [symbol] ) | |
| | AND | |
| | (IF single RR from yesterday > [symbol] ) | |
| | THEN (1 ELSE 0) [symbol] | |
| | Can allow for m [symbol] non-consecutive day(s) of missingness between consecutive readings | |
| L7 | Weekly SpO2 after exertion [over d [symbol] consecutive weeks] | d [symbol] , [symbol] , s [symbol] , |
| | [Condition A] | |
| | (IF week-to-week change < [symbol] ) | |
| | OR | |
| | Condition B] | |
| | [(IF single Sp [symbol] )2 from current week < [symbol] ) | |
| | AND | |
| | (IF single SpO2 from previous week < [symbol] ) | |
| | THEN (1 ELSE 0) [symbol] | |
| | Can allow for m [symbol] non-consecutive week(s) of missingness between consecutive readings | |
| L8 | Weekly Respiratory Rate after exertion [over d [symbol] consecutive weeks] | d [symbol] , [symbol] , s [symbol] , |
| | [Condition A] | |
| | (If change between current (or latest week of data submitted if current is missing) | |
| | week-minus-previous week change > [symbol] ) between all considered weeks | |
| | OR | |
| | Condition B] | |
| | [symbol] F single RR from latest week [symbol] ) | |
| | AND | |
| | (IF single RR from latest week-1 > [symbol] ) | |
| | AND | |
| | (IF single RR from latest week-2 > [symbol] ) | |
| | AND | |
| | (IF single RR from latest week-3 > [symbol] ) | |
| | [symbol] | |
| | THEN (1 ELSE 0) [symbol] | |
| | Can allow for m [symbol] non-consecutive week(s) of missingness between consecutive readings | |
| | but no more than [symbol] missing weeks in a period | |
| L9 | Weekly Pulse Rate after exertion [over d [symbol] consecutive weeks] | d [symbol] , [symbol] , s [symbol] , |
| | [Condition A] | c [symbol] , m [symbol] , [symbol] |
| | (IF change between current (or latest week of data submitted if current is missing) | |
| | week-minus-previous week change > [symbol] ) between all considered weeks | |
| | OR. | |
| | Condition B] | |
| | [(IF single PR from latest week > [symbol] ) | |
| | AND | |
| | (IF single PR from latest week-1 > [symbol] ) | |
| | AND | |
| | (IF single PR from latest week-2 > [symbol] ) | |
| | AND | |
| | (IF single PR from latest week-3 > s [symbol] | |
| | ) | |
| | THEN (1 ELSE 0) [symbol] | |
| | Can allow for non-consecutive week(s) of missingness between consecutive readings | |
| | but no more than [symbol] missing weeks in a period | |
| L10 | Change in active SpO2 relative to resting SpO2 [on day with active and resting | [symbol] |
| | measures] | |
| | [Condition A] | |
| | (IF change in active SpO2 relative to resting SpO2 < [symbol] ) | |
| | THEN (1 ELSE 0) [symbol] | |

RR is respiration rate.
PRO is patient reported outcome.
PR is pulse rate.
SpO₂ is oxygen saturation [symbol]
indicates data missing or illegible when filed

Additional longitudinal rules, L, may be incorporated or only a portion of the above rules may be used. The above longitudinal rules have several factors: d_{L1}, t_{L1}, S_{L1}, c_{L1}, m_{L1}, d_{L2}, t_{L2}, S_{L2}, c_{L2}, m_{L2}, d_{L3}, t_{L3}, S_{L3}, c_{L3}, m_{L3}, d_{L4}, t_{L4}, S_{L4}, c_{L4}, m_{L4}, d_{L5}, t_{L5}, S_{L5}, c_{L5}, m_{L5}, d_{L6}, I_{L6}, S_{L6}, c_{L6}, m_{L6}, d_{L7}, t_{L7}, S_{L7}, c_{L7}, m_{L7}, d_{L8}, t_{L8}, S_{L8}, c_{L8}, m_{L8}, g_{L8}, d_{L9}, t_{L9}, S_{L9}, c_{L9}, m_{L9}, g_{L9}, S_{L10}, and c_{L10}. The value of each of these factors may be in the range of −10 to 150, and any tenth increment therein (e.g., 0.5, 0.6, 0.7, 0.8, 0.9, etc.). In an example, the specific values of each longitudinal factor may be: d_{L1}=2, t_{L1}=2.0, S_{L1}=3.0, c_{L1}=1.0, m_{L1}=1, d_{L2}=2, t_{L2}=2.0, S_{L2}=4.0, c_{L2}=1.0, m_{L2}=1, d_{L3}=2, t_{L3}=2.0, S_{L3}=5.0, c_{L3}=1.0, m_{L3}=1, d_{L4}=2, t_{L4}=−3.0, S_{L4}=93, c_{L4}=1.0, m_{L4}=1, d_{L5}=2, t_{L5}=4, S_{L5}=25, c_{L5}=0.5, m_{L5}=1, d_{L6}=2, t_{L6}=15, S_{L6}=110, c_{L6}=0.5, m_{L6}=1, d_{L7}=2, t_{L7}=−4, S_{L7}=90, c_{L7}=1.0, m_{L7}=1, d_{L8}=4, t_{L8}=4, S_{L8}=25, c_{L8}=0.5, m_{L8}=1, g_{L8}=2, d_{L9}=4, t_{L9}=20, S_{L9}=135, c_{L9}=0.5, m_{L9}=1, g_{L9}=2, S_{L10}=−5, and c_{L10}=1.0.

The longitudinal sum, R_{longitudinal}, may refer to the sum of the outputs from the longitudinal rules. For example, the longitudinal rules may be some or all of the rules provided in L1 to L10 above. In some configurations R_{longitudinal} may include the output of one or more of the baseline rules. For example, R_{longitudinal} may include the sum of the outputs L1 to L10 and B3 of FIG. 6. The longitudinal algorithm may compute whether S plus the longitudinal sum is greater than a threshold value to determine whether there has been a status change in the patient. The algorithm may compute, for example, whether S+R_{longitudinal}>threshold. The threshold value (Sₐₗₐᵣₘ) may be empirically determined for a given indication. The threshold value may be in a range from 0.5 to 10, and any tenth increment therein (e.g., 0.6, 1.4, 7.7, 7.8, 7.9, 9.9, etc.). In a specific example, the threshold value (Sₐₗₐᵣₘ) is 2.5.

At 410, cloud server 102 transmits a notification of the status change of the patient to HCP device 116 based whether S+R_{longitudinal}>threshold. The computed values for S and R_{longitudinal} account for the risk factor the physiological data, and subjective data of the patient before and/or during treatment. The notification may alert the HCP to evaluate the patient's symptoms because the status change indicates signs of lung disease when considering the patient's initial risk factor. In some aspects, cloud server 102 may provide the notification in real-time or near real-time such that the HCP may be alerted of significant status changes and act quickly to evaluate, diagnose, and treat the patient for lung disease. The notification is provided to HCP interface 118. In some aspects, cloud server 102 transmits the physiological data to HCP interface 118 and HCP interface 118 configures the physiological data to be displayed to the HCP. A suspicion of the lung disease may prompt the HCP to take further investigative or interventional (e.g., drug treatment) activity.

The algorithms used herein may be containerized algorithms accessible to and cooperating with an EMR ecosystem.

Based on a clinical trial from 22 ILD cases, an algorithmic solution in accordance with aspects of the invention as described above would have notified the HCP of a change in pulmonary function in >50% of patients at least 14 days (mean: 83, IQR [4-112]) in advance of actual ILD investigation or treatment. This detection of potential ILD in a patient can have significant benefit to the overall patient outcome by enabling early intervention by the HCP.

FIG. 5 shows a computer system 500, according to some aspects. Various aspects and components therein, such as system 100 and/or method 400, can be implemented, for example, using computer system 500 or any other well-known computer systems, such that the computer system, when programmed according to aspects described herein, becomes a special purpose machine.

In some aspects, computer system 500 can comprise one or more processors (also called central processing units, or CPUs), such as a processor 804. Processor 804 can be connected to a communication infrastructure or bus 806.

In some aspects, one or more processors 504 can each be a graphics processing unit (GPU). In some aspects, a GPU is a processor that is a specialized electronic circuit designed to process mathematically intensive applications. The GPU can have a parallel structure that is efficient for parallel processing of large blocks of data, such as mathematically intensive data common to computer graphics applications, images, videos, etc.

In some aspects, computer system 500 can further comprise user input/output device(s) 503, such as monitors, keyboards, pointing devices, etc., that communicate with communication infrastructure 506 through user input/output interface(s) 502. Computer system 500 can further comprise a main or primary memory 508, such as random access memory (RAM). Main memory 508 can comprise one or more levels of cache. Main memory 508 has stored therein control logic (i.e., computer software) and/or data.

In some aspects, computer system 500 can further comprise one or more secondary storage devices or memory 510. Secondary memory 510 can comprise, for example, a hard disk drive 512 and/or a removable storage device or drive 514. Removable storage drive 514 can be a floppy disk drive, a magnetic tape drive, a compact disk drive, an optical storage device, tape backup device, and/or any other storage device/drive. Removable storage drive 514 can interact with a removable storage unit 518. Removable storage unit 518 can comprise a computer usable or readable storage device having stored thereon computer software (control logic) and/or data. Removable storage unit 518 can be a floppy disk, magnetic tape, compact disk, DVD, optical storage disk, and/any other computer data storage device. Removable storage drive 514 reads from and/or writes to removable storage unit 518 in a well-known manner.

In some aspects, secondary memory 510 can comprise other means, instrumentalities or other approaches for allowing computer programs and/or other instructions and/or data to be accessed by computer system 500. Such means, instrumentalities or other approaches can comprise, for example, a removable storage unit 522 and an interface 520. Examples of the removable storage unit 522 and the interface 520 can comprise a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

In some aspects, computer system 500 can further comprise a communication or network interface 524. Communication interface 524 enables computer system 500 to communicate and interact with any combination of remote devices, remote networks, remote entities, etc. (individually and collectively referenced by reference number 528). For example, communication interface 524 can allow computer system 500 to communicate with remote devices 528 over communications path 526, which can be wired and/or wireless, and which can comprise any combination of LANs, WANs, the Internet, etc. Control logic and/or data can be transmitted to and from computer system 500 via communications path 526.

In some aspects, a non-transitory, tangible apparatus or article of manufacture comprising a non-transitory, tangible computer useable or readable medium having control logic (software) stored thereon is also referred to herein as a computer program product or program storage device. This includes, but is not limited to, computer system 500, main memory 508, secondary memory 510, and removable storage units 518 and 522, as well as tangible articles of manufacture embodying any combination of the foregoing. Such control logic, when executed by one or more data processing devices (such as computer system 500), causes such data processing devices to operate as described herein.

In some aspects, one or more methods, systems, or computer readable media as described herein may be provided as a standalone product for patients at risk of ILD. In some aspects, one or more methods, systems, or computer readable media as described herein may be provided as a companion to a pharmaceutical treatment. In some aspects, one or more methods, systems, or computer readable media as described herein may constitute Software as a Medical Device (SaMD).

Based on the teachings contained in this disclosure, it will be apparent to those skilled in the relevant art(s) how to make and use aspects of this disclosure using data processing devices, computer systems and/or computer architectures other than that shown in FIG. 5. In particular, aspects described herein can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

Aspects of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A method for monitoring a patient, the method comprising:
receiving, by one or more processors, patient data collected from the patient prior to the patient beginning a treatment for cancer in the patient, wherein the treatment is determined to increase risk of a lung disease;
identifying, by the one or more processors, a risk factor of lung disease in the patient by providing the patient data to a rule-based algorithm;
while the patient receives the treatment, receiving, by the one or more processors,
physiological data of the patient from a patient device, wherein the physiological data comprises oxygen saturation data measured by a pulse-oximeter; and
subjective patient data from the patient device corresponding to patient-reported experience;
determining, by the one or more processors, a status change of the patient by providing the risk factor of lung disease, the physiological data, and the subjective patient data received during treatment of the patient to a longitudinal algorithm, wherein the status change indicates suspicion of the lung disease in the patient, wherein the status change is based on whether a sum of the risk factor of the patient and output of the longitudinal algorithm exceeds a threshold;
transmitting, by the one or more processors, a notification of the status change of the patient to a health care provider device based.

2. The method of claim 1, wherein the patient data is received from an electronic case report form.

3. The method of claim 1, wherein the patient data comprises at least one of medical history, demographics, or pulmonary function of the patient.

4. The method of claim 1, wherein the physiological data further comprises at least one of resting oxygen saturation, oxygen saturation at exertion, respiratory rate at rest or during or after exertion, and pulse rate.

5. The method of claim 1, wherein the subjective patient data comprises at least one of cough severity, dyspnea severity, chest pain or tightness severity, or health-related quality of life measurements.

6. The method of claim 1, wherein the patient device comprises an application that is configured to transmit the physiological data to a cloud server, wherein the cloud server is executed by the one or more processors.

7. The method of claim 1, wherein the notification is transmitted to the health care provider device in real-time.

8. The method of claim 1, further comprising:
transmitting, by the one or more processors, a data entry notification to the patient device recommending that the patient provide physiological data.

9. The method of claim 1, wherein the lung disease is at least one of:
acute interstitial pneumonitis; acute respiratory distress syndrome; acute respiratory failure; air-space consolidation; allergic eosinophilia; alveolar proteinosis; alveolitis; alveolitis allergic; alveolitis necrotizing; architectural distortion; autoimmune lung disease; bronchiolitis; chronic interstitial (CIP); combined pulmonary fibrosis and emphysema; diffuse alveolar damage; drug-induced interstitial lung disorder; eosinophilia myalgia syndrome; eosinophilic granulomatosis with polyangitis; eosinophilic pneumonia; eosinophilic pneumonia acute; eosinophilic pneumonia chronic; granulomatous pneumonitis; ground-glass attenuation; idiopathic interstitial pneumonia; idiopathic pneumonia syndrome; idiopathic pulmonary fibrosis; interstitial lung disease (ILD); interlobular septal thickening; interstitial pneumonia; interstitial pulmonary disease; intralobular reticular opacity; lung infiltration; lymphangitic carcinomatosis; necrotizing bronchiolitis; nodular opacities; non-septal linear opacity; obliterative bronchiolitis; organizing pneumonia; pleural effusion; pneumonitis; previous tuberculosis; progressive massive fibrosis; pulmonary emphysema; pulmonary fibrosis; pulmonary necrosis; pulmonary radiation injury; pulmonary sarcoidosis; pulmonary toxicity; pulmonary vasculitis; radiation alveolitis; radiation fibrosis; radiation pneumonitis; respiratory failure; restrictive pulmonary disease; rheumatoid lung sarcoidosis; scarring or inflammation of interstitium; small airways disease; thickening of bronchovascular bundles; traction bronchiectasis; transfusion-related acute lung injury; or pneumonia.

10. A system for monitoring a patient, comprising:
a patient device coupled to a pulse-oximeter;
a healthcare provider device; and
a cloud server, comprising:
at least one processor; and
a memory having instructions stored thereon that, when executed by the at least one processor, cause the at least one processor to:
receive patient data collected from the patient prior to the patient beginning treatment for cancer in the patient, wherein the treatment is determined to increase risk of a lung disease;
identify a risk factor of the lung disease in the patient by providing the patient data to a rule-based algorithm;
while the patient receives the treatment, receive, from the patient device,
physiological data of the patient, wherein the physiological data comprises oxygen saturation data measured by the pulse-oximeter;
subjective patient data corresponding to patient-reported experience;
determine a status change of the patient by providing the risk factor of lung disease, the physiological data, and the subjective patient data received during treatment of the patient to a longitudinal algorithm, wherein the status change indicates suspicion of the lung disease in the patient, wherein the status change is based on whether a sum of the risk factor of the patient and output of the longitudinal algorithm exceeds a threshold; and
transmit, to the healthcare provider device, a notification of the status change of the patient.

11. The system of claim 10, wherein the patient data is received from an electronic case report form.

12. The system of claim 10, wherein the patient data comprises at least one of medical history, demographics, or pulmonary function of the patient.

13. The system of claim 10, wherein the physiological data further comprises at least one of resting oxygen saturation, oxygen saturation at exertion, respiratory rate at rest or during or after exertion, and pulse rate.

14. The system of claim 10, wherein the subjective patient data comprises at least one of cough severity, dyspnea severity, chest pain or tightness severity, or health-related quality of life measurements.

15. The system of claim 10, wherein the patient device comprises an application that is configured to transmit the physiological data to the cloud server.

16. The system of claim 10, wherein the notification is transmitted to the health care provider device in real-time.

17. The system of claim 10, wherein the instructions further cause the at least one processor to:
transmit a data entry notification to the patient device recommending that the patient provide physiological data.

18. The system of claim 10, wherein the lung disease is at least one of:
acute interstitial pneumonitis; acute respiratory distress syndrome; acute respiratory failure; air-space consolidation; allergic eosinophilia; alveolar proteinosis; alveolitis; alveolitis allergic; alveolitis necrotizing; architectural distortion; autoimmune lung disease; bronchiolitis; chronic interstitial (CIP); combined pulmonary fibrosis and emphysema; diffuse alveolar damage; drug-induced interstitial lung disorder; eosinophilia myalgia syndrome; eosinophilic granulomatosis with polyangitis; eosinophilic pneumonia; eosinophilic pneumonia acute; eosinophilic pneumonia chronic; granulomatous pneumonitis; ground-glass attenuation; idiopathic interstitial pneumonia; idiopathic pneumonia syndrome; idiopathic pulmonary fibrosis; ILD; interlobular septal thickening; interstitial lung disease; interstitial pneumonia; interstitial pulmonary disease; intralobular reticular opacity; lung infiltration; lymphangitic carcinomatosis; necrotizing bronchiolitis; nodular opacities; non-septal linear opacity; obliterative bronchiolitis; organizing pneumonia; pleural effusion; pneumonitis; previous tuberculosis; progressive massive fibrosis; pulmonary emphysema; pulmonary fibrosis; pulmonary necrosis; pulmonary radiation injury; pulmonary sarcoidosis; pulmonary toxicity; pulmonary vasculitis; radiation alveolitis; radiation fibrosis; radiation pneumonitis; respiratory failure; restrictive pulmonary disease; rheumatoid lung sarcoidosis; scarring or inflammation of interstitium; small airways disease; thickening of bronchovascular bundles; traction bronchiectasis; transfusion-related acute lung injury; or pneumonia.

19. A non-transitory computer-readable medium (CRM) having stored thereon computer-readable instructions executable to cause a computer system to perform operations comprising:
receiving patient data collected from a patient prior to the patient beginning a treatment for cancer in the patient, wherein the treatment is determined to increase risk of a lung disease;
identifying a risk factor of the lung disease in the patient by providing the patient data to a rule-based algorithm;
while the patient receives the treatment, receiving, from a patient device,
physiological data of the patient, wherein the physiological data comprises oxygen saturation data measured by a pulse-oximeter;
subjective patient data from the patient device corresponding to patient-reported experience;
determining a status change of the patient by providing the risk factor of lung disease, the physiological data, and the subjective patient data received during treatment of the patient to a longitudinal algorithm, wherein the status change indicates suspicion of the lung disease in the patient, wherein the status change is based on whether a sum of the risk factor of the patient and output of the longitudinal algorithm exceeds a threshold; and
transmitting, to a healthcare provider device, a notification of the status change of the patient.

20. The computer-readable medium of claim 19, wherein the patient data is received from an electronic case report form.

21. The computer-readable medium of claim 19, wherein the patient data comprises at least one of medical history, demographics, or pulmonary function of the patient.

22. The computer-readable medium of claim 19, wherein the physiological data further comprises at least one of resting oxygen saturation, oxygen saturation at exertion, respiratory rate at rest or during or after exertion, and pulse rate.

23. The computer-readable medium of claim 19, wherein the subjective patient data comprises at least one of cough severity, dyspnea severity, chest pain or tightness severity, or health-related quality of life measurements.

24. The computer-readable medium of claim 19, wherein the patient device comprises an application that is configured to transmit the physiological data to a cloud server, wherein the cloud server is executed by the one or more processors.

25. The computer-readable medium of claim 19, wherein the notification is transmitted to the health care provider device in real-time.

26. The computer-readable medium of claim 19, further comprising:
transmitting, by the one or more processors, a data entry notification to the patient device recommending that the patient provide physiological data.

27. The computer-readable medium of claim 19, wherein the lung disease is at least one of:
acute interstitial pneumonitis; acute respiratory distress syndrome; acute respiratory failure; air-space consolidation; allergic eosinophilia; alveolar proteinosis; alveolitis; alveolitis allergic; alveolitis necrotizing; architectural distortion; autoimmune lung disease; bronchiolitis; CIP; combined pulmonary fibrosis and emphysema; diffuse alveolar damage; drug-induced interstitial lung disorder; eosinophilia myalgia syndrome; eosinophilic granulomatosis with polyangitis; eosinophilic pneumonia; eosinophilic pneumonia acute; eosinophilic pneumonia chronic; granulomatous pneumonitis; ground-glass attenuation; idiopathic interstitial pneumonia; idiopathic pneumonia syndrome; idiopathic pulmonary fibrosis; ILD; interlobular septal thickening; interstitial pneumonia; interstitial pulmonary disease; intralobular reticular opacity; lung infiltration; lymphangitic carcinomatosis; necrotizing bronchiolitis; nodular opacities; non-septal linear opacity; obliterative bronchiolitis; organizing pneumonia; pleural effusion; pneumonitis; previous tuberculosis; progressive massive fibrosis; pulmonary emphysema; pulmonary fibrosis; pulmonary necrosis; pulmonary radiation injury; pulmonary sarcoidosis; pulmonary toxicity; pulmonary vasculitis; radiation alveolitis; radiation fibrosis; radiation pneumonitis; respiratory failure; restrictive pulmonary disease; rheumatoid lung sarcoidosis; scarring or inflammation of interstitium; small airways disease; thickening of bronchovascular bundles; traction bronchiectasis; transfusion-related acute lung injury; or pneumonia.
